(19) United States
(12) Patent Application Publication
(10) Pub. No.: US 2008/0077098 A1
(43) Pub. Date: Mar. 27, 2008

(54) One-Way Valve Apparatus

(76) Inventors: Arthur James Bradley, Red Bluff, CA (US); Donegal Bradley, Red Bluff, CA (US)

Correspondence Address: CENTRAL COAST PATENT AGENCY, INC, 3 HANGAR WAY SUITE D, WATSONVILLE, CA, 95076, US

(21) Appl. No.: 11/862,375

(22) Filed: Sep. 27, 2007

Related U.S. Application Data
(60) Provisional application No. 60/847,985, filed on Sep. 27, 2006.

(51) Int. Cl. A61F 5/442

(52) U.S. Cl. 604/334

(57) ABSTRACT

A one-way valve includes a valve support flange for facilitating attachment of the valve to a host device, a valve tube for facilitating coupling of the valve at one end to a delivery vessel or a delivery system, and a valve sheathing formed substantially tubular and collapsible upon itself, the sheathing open at one end and connected at the other end to the valve tube.

### CROSS-REFERENCE TO RELATED APPLICATIONS

The present invention claims priority to a U.S. provisional patent application Ser. No. 60/847,985 entitled “Evacuable Colostomy Bag with a One-Way Valve”, filed on Sep. 27, 2006, disclosure of which is included herein at least by reference. The present invention is related in part to a U.S. patent application Ser. No. 11/256,301 entitled “METHOD AND APPARATUS FOR FACILITATING EVACUATION AND CLEANSING OF A COLONOSTOMY BAG” filed on Oct. 20, 2005.

### BACKGROUND OF THE INVENTION

1. Field of the Invention

The present invention is in the field of valve apparatus used in such as medical waste management and pertains particularly to a valve apparatus used in evacuating and cleaning medical bags such as colostomy bags used to collect waste matter from patients.

2. Discussion of the State of the Art

In the field of post surgery management of biomedical fluids including waste matter, there is available in the art, a colonostomy, also referred to herein as ostomy or colostomy bag, that may be worn by a user and that is adapted to collect waste matter that has been surgically redirected from the normal path due to a re-routing of the colon during an operational procedure. Most commonly, the colostomy bag is used to collect solid waste from a patient who has undergone a surgical procedure on the colon and is used during in-patient treatment and, most importantly, after the patient has left the place of surgery until sufficient recovery time has elapsed so that a patient's normal function can be re-established.

A problem with use of these types of external bags is a difficulty in evacuating waste from the bag and cleaning the bag for continued use. In prior procedures, the waste collected in the bag is manually coaxed toward a bottom opening in the bag, typically while the bag is attached to its host. Evacuation consists of simply forcing the excrement out of the bottom opening by hand. The bottom opening of the bag is foldable upon it self and closed with a clamp for reuse. Another challenge is cleaning the bottom opening so that it may be refolded and clamped. The entire process is demeaning to some users because of likelihood that their skin may come in contact with their own excrement. Likewise, current procedures do not enable the inside of the bag to be thoroughly cleaned while still attached to the host.

The inventor is aware of a system for top-down evacuating and rinsing of a user-mounted colostomy bag. The system includes a valve assembly affixed through one wall of the colostomy bag, the valve assembly having a first coupling interface and an interiorly protruding stem, at least one solution reservoir for containing an aqueous solution, and a second coupling interface attached directly to or indirectly to at least one of the at least one solution reservoir, the coupling interface for connecting the reservoir to the valve for communication of the aqueous solution from the reservoir to the bag. In a preferred application, the valve assembly of the system is a one way valve.

It has occurred to the inventor that improvements may be made in the architecture of a valve assembly used for delivering solution into a medical bag or some other bag whereby the valve may be implemented with fewer parts and still achieve one-way function during use. Therefore what is clearly needed in the art is a one-way valve apparatus useable for cleaning and other operations whereby solution may be injected into a bag such as a colonostomy bag.

### SUMMARY OF THE INVENTION

A one-way valve includes a valve support flange for facilitating attachment of the valve to a host device, a valve tube for facilitating coupling of the valve at one end to a delivery vessel or a delivery system, and a valve sheathing formed substantially tubular and collapsible upon itself, the sheathing open at one end and connected at the other end to the valve tube.

### BRIEF DESCRIPTION OF THE DRAWING FIGURES

FIG. 1 is a front view of a colostomy bag adapted with a one-way valve according to an embodiment of the present invention.

FIG. 2 is a partial view of the colostomy bag of FIG. 1 further illustrating a syringe coupled to the valve according to an embodiment of the present invention.

FIG. 3 is an elevation view of the valve of FIG. 2 illustrated in an open state.

FIG. 4 is an elevation view of the valve of FIG. 3 illustrated in a closed or collapsed state.

### DETAILED DESCRIPTION

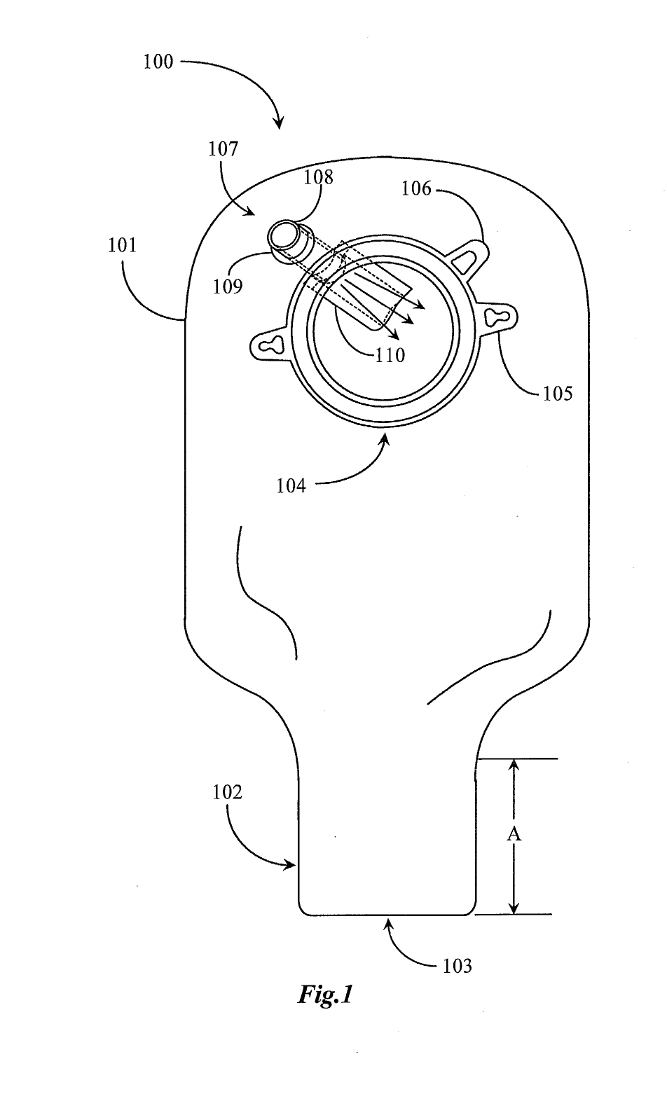

FIG. 1 is a plan view of a colostomy bag 100 according to an embodiment of the present invention. Colostomy bag 100 is adapted as a temporary waste collection bag that is worn externally by a user and at some point discarded after some repeated use. More particularly, bag 100 is adapted as an externally worn system for collecting solid waste from a surgery patient whom has had surgery on the colon and has had function of the colon temporarily, or in some cases, permanently re-routed. Bag 100 is manufactured of a pliable and leak-proof polymer material 101. Material 101 includes 2 opposing thin and pliable sheets of plastic and a soft material sheet (not illustrated) attached on one of the plastic sheets on a side facing the user when mounted. The opposing sides of bag 100 may be glued together or fused via a heating treatment, or a combination of the two. The soft material sheet may also be glued in place and is provided so that the user may be more comfortable when wearing the bag. The soft material sheet serves no other function than to provide a comfortable interface against the skin of a user.

Colostomy bag 100 may be a disposable bag that is typically obtained in a package of several bags. Application of a one-way valve 107 may render the bag less disposable because of the ability to cleanse the bag after use. Bag 100 includes a stoma connector 104 adapted to enable a user to connect bag 100 to like apparatus (not illustrated) attached via tape or via other method over a re-routed stoma (surgical opening). Connector 104 includes an alignment tab 106 and snap flanges 105. Connector 104 is used to secure bag 100 to the associated device attached to the patient's new opening. Connector 104 has an opening centered therein, which is significantly large and adapted to allow solid waste to pass from the patient's colon and re-routed stoma and then into bag 100.

Bag 100 has a bottleneck 102 formed therein at the bottom portion of the bag. Bottleneck portion 102 includes a bottom opening 103 that may be folded over upon itself in order to close the bottom opening of bag 100 to prevent leakage. The length of bottleneck 102 is illustrated as a dimension A, which may be around 2 or 3 inches in length in bags not adapted with a valve for top down cleansing. For colostomy bags adapted for top down evacuation, dimension A may be extended past the typical length for more convenient evacuation such as to enable the bottle neck to reach below a toilet rim while still being worn by the user. Typically, a clamping device (not illustrated) is provided for the purpose of closing the opening at the bottom of bag 100.

As described above, colostomy bag 100 is worn by a user and adapted to receive waste as it passes naturally from the user. After some time of use where waste has been collected by bag 100, a user may desire to evacuate that bag of waste to free the bag for collecting subsequent waste from the user. Colostomy bag 100 serves as a good example of the type of bag that may be adapted with a one-way valve for facilitating top-down cleansing by means of delivering an aqueous solution therein in a manner that may be directionally controlled by the user.

The inventor provides a one-way valve 107 that may be manufactured with or otherwise may be installed to colostomy bag 100. Valve 107 is provided at a strategic location on colostomy bag 100 just above connector 104 and to one side of vertical center of bag 100. In one embodiment, valve 107 includes valve tube 108 and a valve support flange 109. Valve 107 may be installed, in a preferred embodiment, on the side of bag 100 comprising one plastic sheet glued or otherwise attached to a soft material layer. This is the side of bag 100 that comes in contact with a user when worn. The opposite side of bag 100 is a single sheet of plastic. Material 101 described further above encompasses the 3 pliable sheets of material that are glued or otherwise joined along aligned peripheral edges to form the seamed colostomy bag.

Valve 107 further includes a seamed plastic sheet or sheathing 110 that is open at both ends and that is installed at one end over tube 108. Tube 108 may be provided in the form of hard or semi-hard polymer that may be translucent or not. The inner diameter of tube 108 is large enough to cover the end of a tapered nozzle of a solution delivery mechanism such as a plunger-type syringe. The method of introduction of solution into valve 107 is pressure seal.

Valve 107 may be installed by first creating a small opening through the soft material and underlying plastic sheet forming the user-facing side of bag 107. Valve 107 may be inserted partially through the opening from the inside of colostomy bag 107 with a portion of tube 108 with plastic sheathing remaining on the inside of the bag. Flange 109 may be formed as part of tube 108 or it may be installed over tube 108 and glued thereon with an epoxy or fast drying cement. Flange 109 is sufficiently large in diameter such that it covers the created opening through which tube 108 has been inserted. Flange 109 provides a gluing interface for sealing the outside of bag 100 uniformly to tube 108 to form an airtight seal. In one embodiment, a sealing flange similar to flange 109 may also be provided to seal the inside area around the opening and inserted tube 108. Valve 107 may be glued in place using a strong and fast drying epoxy or glue.

The purpose of valve 107 is to provide delivery access to the interior of bag 100 while a patient is wearing it. The internal portion of the bag would otherwise be inaccessible accept for through opening 103 if unclamped.

Valve 107 is adapted for pressure coupling to a system or device capable of delivering an aqueous solution into bag 107. Sheathing 110 provides the one-way valve function for tube 108. The aqueous solution is used to help dissolve any solid waste matter collected in the bag to make it easier to evacuate. The solution can be one of or any mixture of water and one or more agents designed to clean, promote solubility, to disinfect, and so on.

The ability of valve 107 to function as a one-way valve is afforded by the plastic sheathing 110. Sheathing 110 allows solution through to the interior of bag 100 because pressure caused by delivering the solution from the delivery mechanism when interfaced with tube 108 is greater than the pressure inside the bag. The solution passes through plastic sheathing 110 as illustrated by directional arrows. Once the solution is delivered, the pressure on the inside of bag 100 closes sheathing 110 upon itself preventing any gasses, or other material from escaping back through tube 108 thereby accomplishing the one-way valve function.

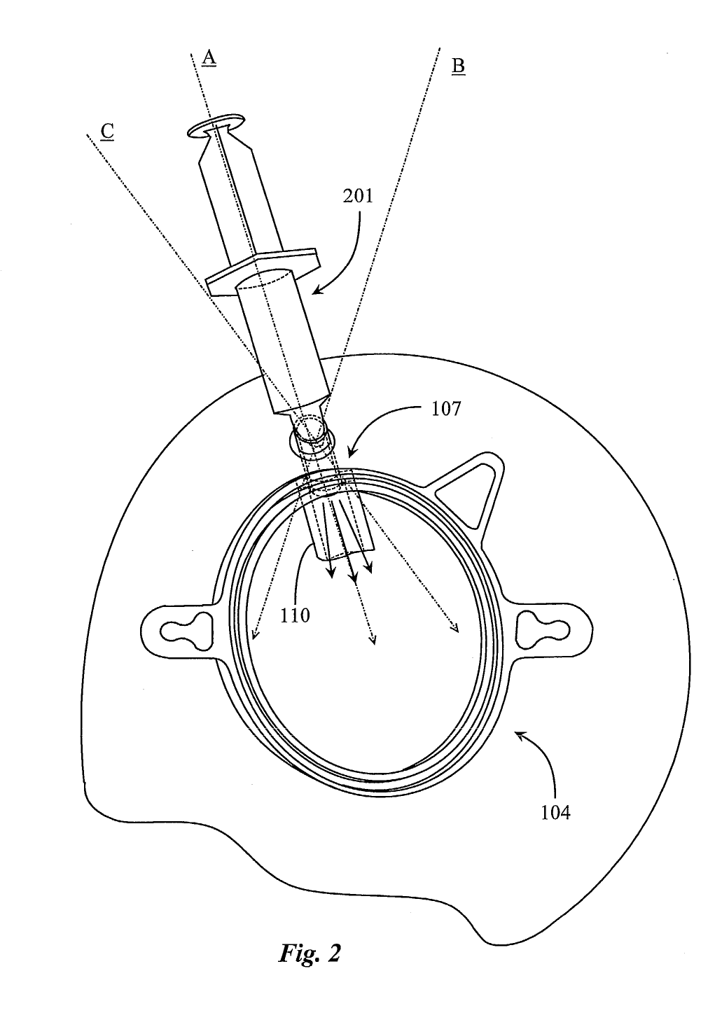

FIG. 2 is a partial view of valve 107 with a solution delivery mechanism 201 coupled thereto according to an embodiment of the invention. Syringe 201 is typical of a plunger type syringe for injecting aqueous solution. The nozzle end of syringe 201 is tapered so that a portion of it is covered by tube 108 such that a pressure seal is formed while pressing the nozzle inside and against the open end of the tube. The tapered outer walls of the nozzle form a seal against the inner wall of tube 108. Other delivery means or mechanisms may be used to couple to valve 107 without departing from the spirit and scope of the present invention. However, the use of a syringe or other handheld delivery vessel facilitates user-controlled direction of the delivery of the solution to specific areas of the inside of the bag.

Using a syringe for delivering the aqueous solution instead of some fixed delivery mechanism enables user control of the direction of the stream of solution as it is being delivered as described immediately above. For example, axis A illustrates general direction of an aqueous stream when syringe 201 is generally pointed in that direction. Axis B and axis C represent two other directional positions that syringe 201 may easily be manipulated to because of the flexible installation of valve 107 onto bag 100 and the fact that delivery is a single axis stream of solution rather than a fixed directional spray as might be the case with a manifold or other multi stream mechanism. Therefore, one advantage of using a valve such as valve 107 is that the user may visually and therefore manually direct the solution to any portion of the upper interior of bag 107 to promote better surface coverage and more uniform cleansing.

Without explicit user control of the direction of the stream of solution into the bag, certain portions of the bag are not reachable and will continue to remain caked with feces presenting an undesirable result. Other types of one-way valves may be employed with bag 100 to enable cleansing with user directional control, however the mechanics of valve 107 are simpler, easier to implement, and are less likely to pose any problems related to solution injection issues such as difficulty coupling the solution delivery means to the valve, or problems related to leak-back that may arise from faulty one-way valve caps or internal valve architecture. Sheathing 110 is seamed on both sides and remains entirely closed and airtight while no solution is being injected effectively preventing back leakage of gasses or materials.

In one embodiment, instead of syringe 201, other forms of solution delivery may be used such as a squeeze-bottle having a tapered nozzle of sufficient diameter to be partially inserted into tube 108 to form a pressure seal. Solution may also be delivered via a delivery hose coupled at one end to a solution reservoir and at the other end to tube 108 of valve 107. In such an implementation, a length of the solution delivery tube end that interfaces with valve 107 may be hard plastic enabling a lever for changing the direction of the stream inside the bag.

In one embodiment of the invention, a standard hose pressure seal connector (not illustrated) could be provided to hold the pressure seal in place whether using a syringe or a delivery hose to connect to the valve tube 108 so that the user does not have to continually apply pressure while introducing the solution into the colostomy bag. Such connectors are available in the art and require only slight modifications in the connecting interfaces such as grooves or the like for retaining clips or spring bars of the connector. The pressure seal connector would typically be installed on the delivery mechanism.

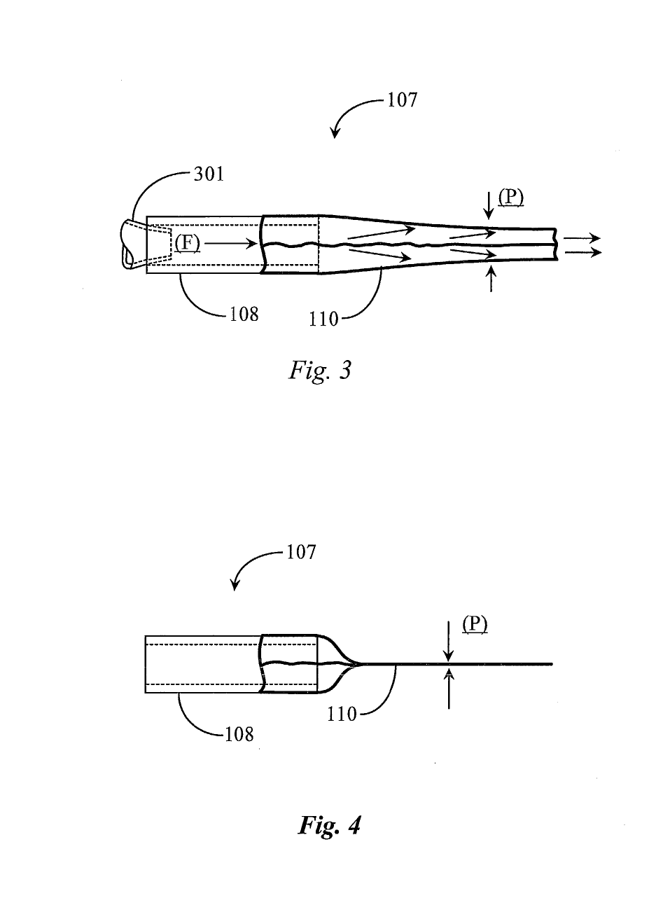

FIG. 3 is an elevation view of valve 107 illustrating an open valve state according to an embodiment of the present invention. Valve 107 is coupled in this example to a tapered nozzle 301 of a solution delivery mechanism such as a syringe or delivery hose. Sheathing 110 is open at both ends and seamed along the sides. One end of sheathing 110 is glued around tube 108 to form an airtight seam between the tube and the sheathing. When a user introduces the solution through valve 107, the solution is forced through tube 108 according to the directional arrow. The force (F) is greater that the pressure (P) on the inside of the colostomy bag normally keeping the sheathing 110 closed in on itself as illustrated by the opposing arrows on either side of sheathing 110.

The solution then is forced through sheathing 110, the walls of the sheathing separating under the force to allow the solution to pass through as illustrated by the directional arrows within sheathing 110.

FIG. 4 is an elevation view of valve 107 of FIG. 3 illustrating a closed valve state according to an embodiment of the present invention.

Valve 107 is closed in on itself in this example due to (P) caused by the airtight colostomy bag as it is used. If no force is introduced as was described above in FIG. 3, then the sheathing 110 remains closed in on itself. The pressure (P) is sufficiently great so as to prevent any gasses or other materials from escaping the bag back through tube 108 thus effectively producing the one-way valve effect. Sheathing 110 opens and closes in on itself accordingly with the introduction of force and the removal of that force. Only a small amount of tube 108 needs to be exposed through the bag wall to interface with the delivery mechanism. Likewise, the total length of the tube with sheathing may be kept short to around about 1 inch or less. A one-way valve such as valve 107 can be manually installed, semi-automatically installed, or automatically installed or formed to a colostomy bag without departing from the spirit and scope of the present invention. Likewise, the materials required to create valve 107 are readily available in the art and may be assembled to form the valve without expensive equipment or process.

It will be apparent to one with skill in the art that the valve apparatus of the invention may be provided using some or all of the mentioned features and components without departing from the spirit and scope of the present invention. It will also be apparent to the skilled artisan that the embodiments described above are exemplary of inventions that may have far greater scope than any of the singular descriptions. For example, a medicine bag for intravenous use may be adapted to include the valve apparatus of the present invention in order to introduce saline or other secondary solutions or medicines periodically without risking leak back of fluids already contained in the bag. Likewise, the valve of the present invention may be used in other applications not related to the medical field such as with inflatable bags or other articles that may be inflated where gas leak back is not desired. There may be many alterations made in the descriptions without departing from the spirit and scope of the present invention.

What is claimed is:

1. A one-way valve comprising:
a valve support flange for facilitating attachment of the valve to a host device;
a valve tube for facilitating coupling of the valve at one end to a delivery vessel or a delivery system; and
a valve sheathing formed substantially tubular and collapsible upon itself, the sheathing open at one end and connected at the other end to the valve tube.
